(19) United States
(12) Patent Application Publication
(10) Pub. No.: US 2006/0142384 A1
(43) Pub. Date: Jun. 29, 2006

(54) 2-Oxyamino-1-cyclopentene-1-nitriles as material protective agents

(76) Inventors: Rainer Bruns, Leverkusen (DE); Oliver Kretschik, Koln (DE); Martin Kugler, Leichlingen (DE); Peter Wachtler, Krefeld (DE)

Correspondence Address: Lanxess Corporation;Law & Intellectual Property Department, 111 Ride Park West Drive, Pittsburgh, PA, 15275-1112, US

(21) Appl. No.: 10/537,243

(22) PCT Filed: Nov. 25, 2003
(86) PCT No.: PCT/EP03/13198

§ 371 Date: Feb. 13, 2006

(30) Foreign Application Priority Data
Dec. 2, 2002 (DE) 102 56 187.7

(51) Int. Cl. A01N 37/34; C07C 255/26

(52) U.S. Cl. 514/519; 558/432; 546/290

(57) ABSTRACT

The novel compounds of the formula (I) 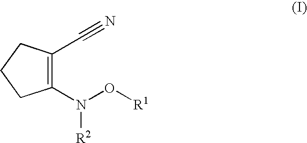 in which
R¹ and R² are as defined in the description are highly suitable for protecting industrial materials against attack and destruction by microorganisms.

The present invention relates to novel 2-oxyamino-1-cyclopentene-1-nitriles, to processes for their preparation, to their use for controlling unwanted microorganisms and to novel mixtures of 2-oxyamino-1-cyclopentene-1-nitriles with other active compounds.

Only very few 2-oxyamino-1-cyclopentene-1-nitriles are already known (cf. V. N. Kopranenkov, B. V. Unkovskii, Izv. Vyssh. Ucheb. Zaved. Khim. Khim. Tekhnol. 1973, 16, 808-9); an activity against material-destroying organisms has hitherto not been described.

This invention now provides novel 2-oxyamino-1-cyclopentene-1-nitriles which, surprisingly, have excellent bactericidal action. By virtue of their antibacterial and antifungal action, the compounds of the general formula (I), individually or as a mixture with one another, are particularly suitable for controlling microorganisms in and on industrial materials.

The present invention provides 2-oxyamino-1-cyclopentene-1-nitriles of the general formula (I), 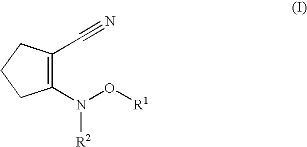
in which
R¹ and R² independently of one another represent hydrogen, halogen, cyano, nitro or represent in each case optionally substituted alkyl, alkenyl, alkynyl, aryl, heterocyclyl or —COR³,
   where
R³ represents in each case optionally substituted alkyl, alkenyl, alkynyl, aryl or heterocyclyl,
   and their salts and acid addition compounds, such as, for example, hydrogenhalides, hydrogenphosphonates or hydrogensulfates.

In the definitions of the substituents R¹ to R³, the saturated and unsaturated hydrocarbon radicals, such as alkyl, alkenyl or alkynyl, are in each case straight-chain or branched, unsubstituted or mono- to polysubstituted by identical or different substituents, including in combination with heteroatoms, such as in alkoxy, haloalkoxy, haloalkylthio or alkylthio, and also in composite terms, such as alkyl- or dialkylamino.

In the alkyl- and dialkylamino substituents mentioned, the alkyl radicals can in each case be identical or different.

Aryl generally represents aromatic mono- or polycyclic hydrocarbon rings which are unsubstituted or mono- to polysubstituted by identical or different substituents, such as, for example, phenyl, naphthyl, anthranyl, phenanthranyl, preferably phenyl or naphthyl, in particular phenyl.

In the terms haloalkyl, haloalkoxy and haloalkylthio, there may in each case be identical or different halogen atoms present. Halogen generally represents fluorine, chlorine, bromine, in particular fluorine or chlorine.

Heterocyclyl generally represents saturated and unsaturated and also aromatic cyclic compounds in which at least one ring member is a heteroatom, i.e. an atom different from carbon, which compounds are unsubstituted or mono- to polysubstituted by identical or different substituents. If the ring contains a plurality of heteroatoms, these can be identical or different. Preferred heteroatoms are oxygen, nitrogen or sulfur. If appropriate, the cyclic compounds form, together with further carbocyclic or heterocyclic fused-on or bridged rings, a polycyclic ring system. A polycyclic ring system may be attached via the heterocyclic ring or via a fused-on carbocyclic ring. Preference is given to mono- or bicyclic ring systems, in particular mono- or bicyclic aromatic ring systems. Preferred heterocyclyl radicals are pyridyl, pyrimidyl, thienyl, furyl and pyrryl.

Preference is given to compounds of the formula (I) in which
R¹ and R² independently of one another represent hydrogen, halogen, cyano, nitro or in each case optionally substituted C₁-C₈-alkyl, C₂-C₈-alkenyl, C₂-C₈-alkynyl, phenyl or heterocyclyl, or represent a radical —COR³,
   where
R³ represents hydrogen, halogen, cyano, nitro or represents in each case optionally substituted C₁-C₈-alkyl, C₂-C₈-alkenyl, C₂-C₈-alkynyl, phenyl or heterocyclyl.

Particular preference is given to compounds of the formula (I) in which
R¹ and R² independently of one another represent hydrogen, halogen, cyano, nitro, or represent C₁-C₈-alkyl, C₂-C₈-alkenyl or C₂-C₈-alkynyl which are in each case optionally mono- or polysubstituted by identical or different substituents from the group consisting of halogen, nitro, cyano, phenyl, C₁-C₆-alkoxy, C₁-C₆-haloalkoxy having 1 to 9 identical or different halogen atoms, C₁-C₆-alkylthio, C₁-C₆-haloalkylthio having 1 to 9 identical or different halogen atoms, C₁-C₆-acyl, C₁-C₆-acyloxy, C₁-C₆-alkoxy-carbonyl or amino, C₁-C₆-alkylamino, di-C₁-C₆-alkylamino, phenylamino or diphenylamino;
   or represent phenyl which is optionally mono- or polysubstituted by identical or different substituents from the group consisting of halogen, cyano, nitro, C₁-C₅-alkyl, C₁-C₅-haloalkyl having 1 to 6 identical or different halogen atoms, C₁-C₅-alkoxy, C₁-C₅-haloalkoxy having 1 to 6 identical or different halogen atoms, C₁-C₅-alkylthio, C₁-C₅-haloalkylthio having 1 to 6 identical or different halogen atoms, amino, C₁-C₆-alkylamino, di-C₁-C₆-alkylamino, phenylamino or diphenylamino;
   or represent heterocyclyl which is optionally mono- or polysubstituted by identical or different substituents from the group consisting of halogen, cyano, nitro, C₁-C₅-alkyl, C₁-C₅-haloalkyl having 1 to 6 identical or different halogen atoms, C₁-C₅-alkoxy, C₁-C₅-haloalkoxy having 1 to 6 identical or different halogen atoms, C₁-C₅-alkylthio, C₁-C₅-haloalkylthio having 1 to 6 identical or different halogen atoms, amino, C₁-C₆-alkylamino or di-C₁-C₆-alkylamino;
   or represent —COR³, where
R³ represents hydrogen, halogen, cyano, nitro, or represents C₁-C₈-alkyl, C₂-C₈-alkenyl, C₂-C₈-alkynyl which are in each case optionally mono- or polysubstituted by identical or different substituents from the group consisting of halogen, nitro, cyano, phenyl, C₁-C₆-alkoxy, C₁-C₆-haloalkoxy having 1 to 9 identical or different halogen atoms, C₁-C₆-alkylthio, C₁-C₆-haloalkylthio having 1 to 9 identical or different halogen atoms, C₁-C₆-acyl, C₁-C₆-acyloxy, C₁-C₆-alkoxy-carbonyl or amino, C₁-C₆-alkylamino, di-C₁-C₆-alkylamino, phenylamino or diphenylamino;
   or represents phenyl which is optionally mono- or polysubstituted by identical or different substituents from the group consisting of halogen, cyano, nitro, C₁-C₅-alkyl, C₁-C₅-haloalkyl having 1 to 6 identical or different halogen atoms, C₁-C₅-alkoxy, C₁-C₅-haloalkoxy having 1 to 6 identical or different halogen atoms, C₁-C₅-alkylthio, C₁-C₅-haloalkylthio having 1 to 6 identical or different halogen atoms, amino, C₁-C₆-alkylamino or di-C₁-C₆-alkylamino;
   or represents heterocyclyl which is optionally mono- or polysubstituted by identical or different substituents from the group consisting of halogen, cyano, nitro, C₁-C₅-alkyl, C₁-C₅-haloalkyl having 1 to 6 identical or different halogen atoms, C₁-C₅-alkoxy, C₁-C₅-haloalkoxy having 1 to 6 identical or different halogen atoms, C₁-C₅-alkylthio, C₁-C₅-haloalkylthio having 1 to 6 identical or different halogen atoms, amino, C₁-C₆-alkylamino or di-C₁-C₆-alkylamino.

Very particular preference is given to compounds of the formula (I) in which
R¹ and R² independently of one another represent hydrogen, fluorine, chlorine, bromine, cyano, nitro or represent C₁-C₆-alkyl, C₂-C₆-alkenyl, C₂-C₆-alkynyl which are in each case optionally mono- to tetrasubstituted by identical or different substituents from the group consisting of fluorine, chlorine, bromine, nitro, cyano, phenyl, C₁-C₄-alkoxy, C₁-C₄-haloalkoxy having 1 to 7 identical or different fluorine, chlorine or bromine atoms, C₁-C₄-alkylthio, C₁-C₄-haloalkylthio having 1 to 7 identical or different fluorine, chlorine or bromine atoms, C₁-C₄-acyl, C₁-C₄-acyloxy, C₁-C₄-alkoxy-carbonyl, amino, C₁-C₄-alkylamino, di-C₁-C₄-alkylamino, phenylamino or diphenylamino;
   or represent phenyl which is optionally mono- to tetrasubstituted by identical or different substituents from the group consisting of fluorine, chlorine, bromine, cyano, nitro, C₁-C₄-alkyl, C₁-C₄-haloalkyl having 1 to 4 identical or different fluorine, chlorine or bromine atoms, C₁-C₄-alkoxy, C₁-C₄-haloalkoxy having 1 to 4 identical or different fluorine, chlorine or bromine atoms, C₁-C₄-alkylthio, C₁-C₄-haloalkylthio having 1 to 4 identical or different fluorine, chlorine or bromine atoms, amino, C₁-C₄-alkylamino or di-C₁-C₄-alkylamino;
   or represent heterocyclyl which is optionally mono- to tetrasubstituted by identical or different substituents from the group consisting of fluorine, chlorine, bromine, cyano, nitro, C₁-C₄-alkyl, C₁-C₄-haloalkyl having 1 to 4 identical or different fluorine, chlorine or bromine atoms, C₁-C₄-alkoxy, C₁-C₄-haloalkoxy having 1 to 4 identical or different fluorine, chlorine or bromine atoms, C₁-C₄-alkylthio, C₁-C₄-haloalkylthio having 1 to 4 identical or different fluorine, chlorine or bromine atoms, amino, C₁-C₄-alkylamino or di-C₁-C₄-alkylamino;
   or represent —COR³, where
R³ represents hydrogen, fluorine, chlorine, bromine, cyano, nitro or represents C₁-C₆-alkyl, C₂-C₆-alkenyl, C₂-C₆-alkynyl which are in each case optionally mono- to tetrasubstituted by identical or different substituents from the group consisting of fluorine, chlorine, bromine, nitro, cyano, phenyl, C₁-C₄-alkoxy, C₁-C₄-haloalkoxy having 1 to 7 identical or different fluorine, chlorine or bromine atoms, C₁-C₄-alkylthio, C₁-C₄-haloalkylthio having 1 to 7 identical or different fluorine, chlorine or bromine atoms, C₁-C₄-acyl, C₁-C₄-acyloxy, C₁-C₄-alkoxy-carbonyl, amino, C₁-C₄-alkylamino, di-C₁-C₄-alkylamino, phenylamino or diphenylamino;
   or represents phenyl which is optionally mono- to tetrasubstituted by identical or different substituents from the group consisting of fluorine, chlorine, bromine, cyano, nitro, C₁-C₄-alkyl, C₁-C₄-haloalkyl having 1 to 4 identical or different fluorine, chlorine or bromine atoms, C₁-C₄-alkoxy, C₁-C₄-haloalkoxy having 1 to 4 identical or different fluorine, chlorine or bromine atoms, C₁-C₄-alkylthio, C₁-C₄-haloalkylthio having 1 to 4 identical or different fluorine, chlorine or bromine atoms, amino, C₁-C₄-alkylamino or di-C₁-C₄-alkylamino;
   or represents heterocyclyl which is optionally mono- to tetrasubstituted by identical or different substituents from the group consisting of fluorine, chlorine, bromine, cyano, nitro, C₁-C₄-alkyl, C₁-C₄-haloalkyl having 1 to 4 identical or different fluorine, chlorine or bromine atoms, C₁-C₄-alkoxy, C₁-C₄-haloalkoxy having 1 to 4 identical or different fluorine, chlorine or bromine atoms, C₁-C₄-alkylthio, C₁-C₄-haloalkylthio having 1 to 4 identical or different fluorine, chlorine or bromine atoms, amino, C₁-C₄-alkylamino or di-C₁-C₄-alkylamino.

Especially preferred are compounds of the formula (I) in which
R¹ and R² independently of one another represent hydrogen, or represent C₁-C₄-alkyl, C₂-C₄-alkenyl, C₂-C₄-alkynyl which are in each case optionally mono- to trisubstituted by identical or different substituents from the group consisting of fluorine, chlorine, bromine, nitro, cyano, or phenyl;
   or represent phenyl which is optionally mono- to disubstituted by identical or different substituents from the group consisting of fluorine, chlorine, bromine, cyano, nitro, C₁-C₂-alkyl, C₁-haloalkyl having 1 to 3 identical or different fluorine or chlorine atoms, amino, monomethylamino, or dimethylamino;
   or represent heterocyclyl which is optionally mono- to disubstituted by identical or different substituents from the group consisting of fluorine, chlorine, bromine, cyano, nitro, C₁-C₂-alkyl, C₁-haloalkyl having 1 to 3 identical or different fluorine or chlorine atoms, amino, monomethylamino, or dimethylamino;
   or represent —COR³, where
R³ represents hydrogen, or represents C₁-C₅-alkyl, C₂-C₅-alkenyl, C₂-C₅-alkynyl which are in each case optionally mono- to trisubstituted by identical or different substituents from the group consisting of fluorine, chlorine, bromine, nitro, cyano, or phenyl;
   or represents phenyl which is optionally mono- to disubstituted by identical or different substituents from the group consisting of fluorine, chlorine, bromine, cyano, nitro, C₁-C₂-alkyl, C₁-haloalkyl having 1 to 3 identical or different fluorine or chlorine atoms, methoxy, amino, monomethylamino, or dimethylamino;
   or represents heterocyclyl which is optionally mono- to disubstituted by identical or different substituents from the group consisting of fluorine, chlorine, bromine, cyano, nitro, C₁-C₂-alkyl, C₁-haloalkyl having 1 to 3 identical or different fluorine or chlorine atoms, amino, monomethylamino, or dimethylamino.

Especially preferred are furthermore compounds of the formula (I) in which
R¹ and R² independently of one another represent hydrogen, C₁-C₅-alkyl, or represent benzyl, 4-methylbenzyl, 4-chlorobenzyl, 4-fluorobenzyl, 4-methoxybenzyl;
   or represent phenyl which is optionally substituted by fluorine, chlorine, alkyl, C₁-haloalkyl having 1 to 3 identical or different fluorine or chlorine atoms;
   or represent —COR³, where
R³ represents C₁-C₅-alkyl which is optionally mono- to trisubstituted by identical or different substituents from the group consisting of fluorine and chlorine;
   or represents phenyl which is optionally substituted by fluorine, chlorine, methoxy, alkyl, C₁-haloalkyl having 1 to 3 identical or different fluorine or chlorine atoms;
   or represents 2-furyl or 2-thienyl which are in each case optionally substituted by methyl, fluorine or chlorine.

The radical definitions given in the respective combinations or preferred and particularly preferred and especially preferred combinations of radicals specifically for these radicals are, independently of the combination given in each case, also replaced by radical definitions of other combinations. Moreover, it is also possible for radical definitions from each preferred range not to apply.

The compounds of the general formula (I) 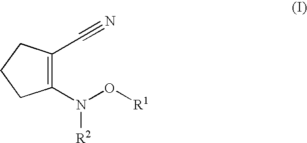
in which R¹ and R² are as defined above
except for compounds (I) in which R¹ and R² are identical and represent ═COR³—
   can be prepared
   by reacting 2-amino-1-cyclopentene-1-carbonitrile of the formula 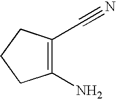
   with hydroxylamines of the general formula (II) 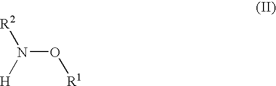
   or salts thereof,
   in which R¹ and R² are as defined above,
   but R¹ and R² do not simultaneously represent —COR³,
   if appropriate in the presence of diluents and if appropriate in the presence of a catalytic or stoichiometric amount of base.

Suitable diluents which may be added, if appropriate, are both water and all customary organic solvents. These preferably include alcohols, such as ethanol or propanol, hydrocarbons, such as toluene, xylene or hexane, chlorinated hydrocarbons, such as chlorobenzene, methylene chloride or chloroform, ketones, such as acetone or butanone, ethers, such as tetrahydrofuran, diethyl ether, methyl tert-butyl ether, dimethoxyethane or dioxane, nitriles, such as acetonitrile, amides, such as N,N-dimethylformamide, N,N-dimethylacetamide or N-methylpyrrolidone, sulfoxides, such as dimethyl sulfoxide, sulfones, such as sulfolane, and also esters, such as ethyl acetate or methyl acetate. The solvents can be employed on their own or in any mixture with one another.

Suitable bases are weak organic bases, such as, for example, tertiary amines, preferably triethylamine, diethylamine, dimethylpyridine and pyridine, or mixtures of these.

The reaction temperature in the preparation process can be varied within a wide temperature range. In general, the process is carried out at temperatures between −30° C. and +150° C., preferably between 0° C. and +110° C.

The preparation of 2-amino-1-cyclopentene-1-carbonitrile has already been disclosed in the literature (cf. Q. E. Thompson., J. Am. Chem. Soc. 1958, 80, 5483-5487).

The compounds of the general formula (I) 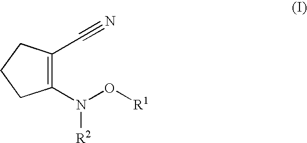
in which R¹ and R² are identical and represent —COR³
can be prepared
by reacting 5,6-dihydro-4H-cyclopenta[c]isoxazol-3-amine of the formula 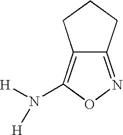
with carbonyl chlorides of the general formula (III) 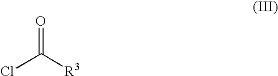
in which R³ is as defined above,
if appropriate in the presence of diluents and if appropriate in the presence of a catalytic or stoichiometric amount of base.

Suitable diluents which may be added, if appropriate, are both water and all customary organic solvents. These preferably include alcohols, such as ethanol or propanol, hydrocarbons, such as toluene, xylene or hexane, chlorinated hydrocarbons, such as chlorobenzene, methylene chloride or chloroform, ketones, such as acetone or butanone, ethers, such as tetrahydrofuran, diethyl ether, methyl tert-butyl ether, dimethoxyethane or dioxane, nitriles, such as acetonitrile, amides, such as NN-dimethylformamide, N,N-dimethylacetamide or N-methylpyrrolidone, sulfoxides, such as dimethyl sulfoxide, sulfones, such as sulfolane, and also esters, such as ethyl acetate or methyl acetate. The solvents can be employed on their own or in any mixture with one another.

Suitable bases are weak organic bases, such as, for example, tertiary amines, preferably triethylamine, diethylamine, dimethylpyridine and pyridine, or mixtures of these.

The reaction temperature in the preparation process can be varied within a wide temperature range. In general, the process is carried out at temperatures between −30° C. and +150° C., preferably between 0° C. and +110° C.

5,6-Dihydro-4H-cyclopenta[c]isoxazole-3-amine can be prepared by reacting 2-amino-1-cyclopentene-1-carbonitrile with hydroxylamine hydrochloride, followed by reaction with aqueous sodium hydroxide solution.

The compounds of the general formula (I) according to the invention have strong herbicidal action and can be used for controlling unwanted microorganisms, such as fungi and bacteria, in crop protection and in the protection of materials.

The invention also provides the use of the compounds of the formula (I) according to the invention as microbicides for protecting industrial materials.

In the protection of materials, the compounds according to the invention can be used for protecting industrial materials against attack and destruction by unwanted microorganisms. In the present context, industrial materials are to be understood as meaning non-living materials which have been prepared for use in industry. Industrial materials which are to be protected by active compounds according to the invention against microbial change or destruction are, for example, adhesives, sizes, paper and board, textiles, leather, wood, timber products, paints and synthetic articles, cooling lubricants and other materials which can be attacked or destroyed by microorganisms. Parts of production plants, for example cooling-water circuits, which may be impaired by the multiplication of microorganisms may also be mentioned as materials to be protected. Industrial materials in the context of the present invention are preferably adhesives, sizes, paper and board, leather, wood, paints, cooling lubricants and heat transfer liquids.

Examples of microorganisms which are capable of bringing about degradation of, or change in, the industrial materials and which may be mentioned are bacteria, fungi, yeast, algae and slime organisms. The active compounds according to the invention preferably act against fungi, in particular molds, wood-discoloring and wood-destroying fungi (Basidiomycetes) and also against slime organisms and bacteria.

Microorganisms of the following genera may be mentioned by way of example:
Alternuria, such as Alternaria tenuis,
Aspergillus, such as Aspergillus niger,
Chaetomium, such as Chaetomium globosum,
Coniophora, such as Coniophora puetana,
Lentinus, such as Lentinus tigrinus,
Penicillium, such as Penicillium glaucum,
Polyporus, such as Polyporus versicolor,
Aureobasidium, such as Aureobasidium pullulans,
Sclerophoma, such as Sclerophoma pityophila,
Trichoderma, such as Trichoderma viride,
Escherichia, such as Escherichia coli,
Pseudomonas, such as Pseudomonas aeruginosa,
Staphylococcus, such as Staphylococcus aureus.

The compounds (I) according to the invention can be used on their own or in any mixture with one another for protecting industrial materials. Depending on their respective physical and/or chemical properties, the compounds of the invention or their mixtures can furthermore be converted into customary formulations, such as solutions, emulsions, suspensions, powders, foams, pastes, granules, aerosols and also very fine capsules in polymeric substances.

These formulations can be prepared in a known manner, for example by mixing the individual active compounds with extenders, that is, liquid solvents, liquefied gases under pressure, and/or solid carriers, if appropriate with the use of surfactants, that is emulsifiers and/or dispersants and/or foam-formers. If the extender used is water, it is also possible to use for example organic solvents as auxiliary solvents. Essentially, suitable liquid solvents are: aromatics, such as xylene, toluene or alkylnaphthalenes, chlorinated aromatics or chlorinated aliphatic hydrocarbons, such as chlorobenzenes, chloroethylenes or methylene chloride, aliphatic hydrocarbons, such as cyclohexane or paraffins, for example mineral oil fractions, alcohols, such as butanol or glycol and their ethers and esters, ketones, such as acetone, methyl ethyl ketone, methyl isobutyl ketone or cyclohexanone, strongly polar solvents, such as dimethylformamide and dimethyl sulfoxide, and water. By liquefied gaseous extenders or carriers are meant liquids which are gaseous at ambient temperature and under atmospheric pressure, for example aerosol propellants, such as halogenated hydrocarbons and butane, propane, nitrogen and carbon dioxide. Suitable solid carriers are: for example ground natural minerals, such as kaolins, clays, talc, chalk, quartz, attapulgite, montmorillonite or diatomaceous earth, and ground synthetic minerals, such as finely divided silica, alumina and silicates. Suitable solid carriers for granules are: for example crushed and fractionated natural rocks such as calcite, marble, pumice, sepiolite and dolomite, and synthetic granules of organic and inorganic meals, and granules of organic material such as sawdust, coconut shells, maize cobs and tobacco stalks. Suitable emulsifiers and/or foam-formers are: for example nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates and protein hydrolyzates. Suitable dispersants are: for example lignosulfite waste liquors and methylcellulose.

Tackifiers such as carboxymethylcellulose and natural and synthetic polymers in the form of powders, granules or latices, such as gum arabic, polyvinyl alcohol and polyvinyl acetate, and natural phospholipids, such as cephalins and lecithins, and synthetic phospholipids, can be used in the formulations. Other possible additives are mineral and vegetable oils.

It is possible to use colorants such as inorganic pigments, for example iron oxide, titanium oxide and Prussian blue, and organic dyestuffs, such as alizarin dyestuffs, azo dyestuffs and metal phthalocyanin dyestuffs, and trace nutrients such as salts of iron, manganese, boron, copper, cobalt, molybdenum and zinc.

The formulations generally comprise between 0.1 and 95% by weight of active compound or active compound mixture, preferably between 2 and 75% by weight.

The present invention furthermore provides microbicidal compositions based on the compounds according to the invention, which compositions comprise at least one solvent or diluent and, if appropriate, processing auxiliaries and, if appropriate, further antimicrobially active compounds.

The efficacy and the activity spectrum of the active compounds of the formula (I) and of the compositions preparable therefrom, of precursors or of formulations in general can be increased by adding, if appropriate, further antimicrobial compounds, fungicides, bactericides, herbicides, insecticides or other active compounds, so as to widen the spectrum of activity or to obtain particular effects such as, for example, additional protection against insects. These mixtures may have a wider activity spectrum than the compounds according to the invention.

In many cases, synergistic effects are obtained, i.e. the activity of the mixture is greater than the activity of the individual components. The following co-components are found to be particularly favorable:

triazoles such as:

azaconazole, azocyclotin, bitertanol, bromuconazole, cyproconazole, diclobutrazole, difenoconazole, diniconazole, epoxyconazole, etaconazole, fenbuconazole, fenchlorazole, fenethanil, fluquinconazole, flusilazole, flutriafol, furconazole, hexaconazole, imibenconazole, ipconazole, isozofos, myclobutanil, metconazole, paclobutrazol, penconazole, propioconazole, prothioconazole, simeoconazole, (±)-cis-1-(4-chlorophenyl)-2-(1H-1,2,4-triazol-1-yl)cycloheptanol, 2-(1-tert-butyl)-1-(2-chlorophenyl)-3-(1,2,4-triazol-1-yl)propan-2-ol, tebuconazole, tetraconazole, triadimefon, triadimenol, triapenthenol, triflumizole, triticonazole, uniconazole and their metal salts and acid adducts;
   imidazoles such as:
clotrimazole, bifonazole, climbazole, econazole, fenapamil, imazalil, isoconazole, ketoconazole, lombazole, miconazole, pefurazoate, prochloraz, triflumizole, thiazolcar, 1-imidazolyl-1-(4′-chlorophenoxy)-3,3-dimethylbutan-2-one, and their metal salts and acid adducts;
   pyridines and pyrimidines such as:
ancymidol, buthiobate, fenarimol, mepanipyrin, nuarimol, pyroxyfur, triamirol;
   succinate dehydrogenase inhibitors such as:
benodanil, carboxim, carboxim sulfoxide, cyclafluramid, fenfuram, flutanil, furcarbanil, furmecyclox, mebenil, mepronil, methfuroxam, metsulfovax, nicobifen, pyrocarbolid, oxycarboxin, Shirlan, Seedvax;
   naphthalene derivatives such as:
terbinafine, naftifine, butenafine, 3-chloro-7-(2-aza-2,7,7-trimethyloct-3-en-5-yne);
   sulfenamides such as:
dichlorfluanid, tolylfluanid, folpet, fluorofolpet; captan, captofol;
   benzimidazoles such as:
carbendazim, benomyl, fuberidazole, thiabendazole or their salts;
   morpholine derivatives such as:
aldimorph, dimethomorph, dodemorph, falimorph, fenpropidin fenpropimorph, tridemorph, trimorphamid and their arylsulfonate salts such as, for example, p-toluenesulfonic acid and p-dodecylphenylsulfonic acid;
   benzothiazoles such as:
2-mercaptobenzothiazole;
   benzothiophene dioxides such as:
N-cyclohexyl-benzo[b]thiophenecarboxamide S,S-dioxide;
   benzamides such as:
2,6-dichloro-N-(4-trifluoromethylbenzyl)-benzamide, tecloftalam;
   boron compounds such as:
boric acid, boric ester, borax;
   formaldehyde and formaldehyde-releasing compounds such as:
benzyl alcohol mono-(poly)-hemiformal, n-butanol hemiformal, dazomet, ethylene glycol hemiformal, hexahydro-5-triazine, hexamethylenetetramine, N-hydroxymethyl-N′-methylthiourea, N-methylolchloroacetamide, oxazolidine, paraformaldehyde, taurolin, tetrahydro-1,3-oxazine, N-(2-hydroxypropyl)aminemethanol, tetramethyloylacetylenediurea;
   isothiazolinones such as:
N-methylisothiazolin-3-one, 5-chloro-N-methylisothiazolin-3-one, 4,5-dichloro-N-octylisothiazolin-3-one, 5-chloro-N-octylisothiazolinone, N-octylisothiazolin-3-one, 4,5-trimethyleneisothiazolinone, 4,5-benzoisothiazolinone;
   aldehydes such as:
cinnamaldehyde, formaldehyde, glutardialdehyde, β-bromocinnamaldehyde, o-phthaldialdehyde;
   thiocyanates such as:
thiocyanatomethylthiobenzothiazole, methylene bisthiocyanate;
   quaternary ammonium compounds and guanidine such as:
benzalkonium chloride, benzyldimethyltetradecylammonium chloride, benzyldimethyldodecylammonium chloride, dichlorobenzyldimethylalkylammonium chloride, didecyldimethylammonium chloride, dioctyldimethylammonium chloride, N-hexadecyltrimethylammonium chloride, 1-hexadecylpyridinium chloride, iminoctadine tris(albesilate);
   iodine derivatives such as:
diiodomethyl p-tolyl sulfone, 3-iodo-2-propynyl alcohol, 4-chlorophenyl-3-iodo-propargylformal, 3-bromo-2,3-diiodo-2-propenyl ethylcarbamate, 2,3,3-triiodoallyl alcohol, 3-bromo-2,3-diiodo-2-propenyl alcohol, 3-iodo-2-propynyl n-butyl-carbamate, 3-iodo-2-propynyl n-hexylcarbamate, 3-iodo-2-propynyl cyclohexylcarbamate, 3-iodo-2-propynyl phenylcarbamate;
   phenols such as:
tribromophenol, tetrachlorophenol, 3-methyl-4-chlorophenol, 3,5-dimethyl-4-chlorophenol, dichlorophene, 2-benzyl-4-chlorophenol, triclosan, diclosan, hexachlorophene, p-hydroxybenzoates, o-phenylphenol, m-phenylphenol, p-phenylphenol, 4-(2-tert-butyl-4-methylphenoxy)phenol, 4-(2-isopropyl-4-methylphenoxy)phenol, 4-(2,4-dimethylphenoxy)phenol and their alkali metal salts and alkaline earth metal salts;
   microbicides with an activated halogen group such as:
bronopol, bronidox, 2-bromo-2-nitro-1,3-propanediol, 2-bromo-4′-hydroxyacetophenone, 1-bromo-3-chloro-4,4,5,5-tetramethyl-2-imidazolidinone, β-bromo-β-nitrostyrene, chloracetamide, chloramine T, 1,3-dibromo-4,4,5,5-tetramethyl-2-imidazolidinone, dichloramine T, 3,4-dichloro-(3H)-1,2-dithiol-3-one, 2,2-dibromo-3-nitrilopropionamide, 1,2-dibromo-2,4-dicyanobutane, halane, halazone, mucochloric acid, phenyl (2-chlorocyanovinyl) sulfone, phenyl (1,2-dichloro-2-cyanovinyl) sulfone, trichloroisocyanuric acid;
   pyridines such as:
1-hydroxy-2-pyridinethione (and their Cu, Na, Fe, Mn, Zn salts), tetrachloro-4-methylsulfonylpyridine, pyrimethanol, mepanipyrim, dipyrithione, 1-hydroxy-4-methyl-6-(2,4,4-trimethylpentyl)-2(1H)-pyridine;
   methoxyacrylates or similar such as:
azoxystrobin, dimoxystrobin, fluoxastrobin, kresoxim-methyl, metominostrobin, orysastrobin, picoxystrobin, pyraclostrobin, trifloxystrobin, 2,4-dihydro-5-methoxy-2-methyl-4-[2-[[[[1-[3-(trifluoromethyl)phenyl]ethylidene]amino]oxy]-methyl]phenyl]-3H-1,2,4-triazol-3-one (CAS-No. 185336-79-2);
   metal soaps such as:
salts of the metals tin, copper and zinc with higher fatty acids, resin acids, naphthenoic acids and phosphoric acids such as, for example, tin naphthenate, tin octoate, tin 2-ethylhexanoate, tin oleate, tin phosphate, tin benzoate, copper naphthenate, copper octoate, copper 2-ethylhexanoate, copper oleate, copper phosphate, copper benzoate, zinc naphthenate, zinc octoate, zinc 2-ethylhexanoate, zinc oleate, zinc phosphate, zinc benzoate;
   metal salts such as:
salts of the metals tin, copper, zinc, and also chromates and dichromates, such as, for example, copper hydroxycarbonate, sodium dichromate, potassium dichromate, potassium chromate, copper sulfate, copper chloride, copper borate, zinc fluorosilicate, copper fluorosilicate;
   oxides such as:
oxides of the metals tin, copper and zinc, such as, for example, tributyltin oxide, Cu₂O, CuO, ZnO;
   oxidizing agents such as:
hydrogen peroxide, peracetic acid, potassium persulfate;
   dithiocarbamates such as:
cufraneb, ferban, potassium N-hydroxymethyl-N′-methyldithiobarbamate, sodium dimethyldithiocarbamate, potassium dimethyldithiocarbamate, macozeb, maneb, metam, metiram, thiram, zineb, ziram;
   nitriles such as:
2,4,5,6-tetrachloroisophthalonitrile, disodium cyano-dithioimidocarbamate;
   quinolines such as:
8-hydroxyquinoline and their copper salts;
   other fungicides and bactericides such as:
bethozaxin, 5-hydroxy-2(5H)-furanone, 4,5-benzodithiazolinone, 4,5-trimethylenedithiazolinone, N-(2-p-chlorobenzoylethyl)-hexaminium chloride, 2-oxo-2-(4-hydroxyphenyl)acetohydroxycinnamoyl chloride, tris-N-(cyclohexyldiazeniumdioxy)-aluminum, N-(cyclo-hexyldiazeniumdioxy)-tributyltin or its potassium salts, bis-N-(cyclohexyldiazeniumdioxy)-copper, iprovalicarb, fenhexamide, spiroxamine, carpropamid, diflumetorin, quinoxyfen, famoxadone, polyoxorim, acibenzolar S-methyl, furametpyr, thifluzamide, methalaxy-M, benthiavalicarb, metrafenon, cyflufenamid, tiadinil, tea tree oil, phenoxyethanol,

Ag, Zn or Cu-containing zeolites alone or incorporated into polymeric materials.

Very especially preferred are mixtures with
azaconazole, bromuconazole, cyproconazole, dichlobutrazol, diniconazole, hexaconazole, metaconazole, penconazole, propiconazole, tebuconazole, dichlofluanid, tolylfluanid, fluorfolpet, methfuroxam, carboxin, benzo[b]thiophene S,S-dioxide cyclohexylcarboxamide, fenpiclonil, 4-(2,2-difluoro-1,3-benzodioxol-4-yl)-1H-pyrrole-3-carbonitrile, butenafine, imazalil, N-methyl-isothiazolin-3-one, 5-chloro-N-methylisothiazolin-3-one, N-octylisothiazolin-3-one, dichloro-N-octylisothiazolinone, mercaptobenthiazole, thiocyanatomethylthiobenzothiazole, benzoisothiazolinone, N-(2-hydroxypropyl)-amino-methanol, benzyl alcohol (hemi)-formal, N-methylolchloroacetamide, N-(2-hydroxypropyl)-amino-methanol, glutaraldehyde, omadine, dimethyl dicarbonate, 2-bromo-2-nitro-1,3-propanediol and/or 3-iodo-2-propynyl n-butylcarbamate, bethoxazin, o-phthaldialdehyde.

Apart from with the abovementioned fungicides and bactericides, mixtures with a good efficacy are, moreover, also prepared with other active compounds:

insecticides/acaricides/nematicides:

abamectin, acephate, acetamiprid, acetoprole, acrinathrin, alanycarb, aldicarb, aldoxycarb, aldrin, allethrin, alpha-cypermethrin, amidoflumet, amitraz, avermectin, azadirachtin, azinphos A, azinphos M, azocyclotin,
Bacillus thuringiensis, barthrin, 4-bromo-2(4-chlorophenyl)-1-(ethoxymethyl)-5-(tri-fluoromethyl)-1H-pyrrole-3-carbonitrile, bendiocarb, benfuracarb, bensultap, beta-cyfluthrin, bifenthrin, bioresmethrin, bioallethrin, bistrilfluron, bromophos A, bromophos M, bufencarb, buprofezin, butathiophos, butocarboxin, butoxycarboxim, cadusafos, carbaryl, carbofuran, carbophenothion, carbosulfan, cartap, quinomethionate, cloethocarb, 4-chloro-2-(2-chloro-2-methylpropyl)-5-[(6-iodo-3-pyridinyl)methoxy]-3(2H)-pyridazinone (CAS-RN: 120955-77-3), chlordane, chlorethoxyfos, chlorfenapyr, chlorfenvinphos, chlorfluazuron, chlormephos, N-[(6-chloro-3-pyridinyl)-methyl]-N′-cyano-N-methyl-ethaneimidamide, chlorpicrin, chlorpyrifos A, chlorpyrifos M, cis-resmethrin, clocythrin, clothiazoben, cypophenothrin clofentezin, coumaphos, cyanophos, cycloprothrin, cyfluthrin, cyhalothrin, cyhexatin, cypermethrin, cyromazin,
decamethrin, deltamethrin, demeton M, demeton S, demeton-5-methyl, diafenthiuron, dialiphos, diazinon, 1,2-dibenzoyl-1(1,1-dimethyl)-hydrazine, DNOC, dichlofenthion, dichlorvos, dicliphos, dicrotophos, difethialone, diflubenzuron, dimethoate, 3,5-dimethylphenyl methylcarbamate, dimethyl-(phenyl)-silyl-methyl-3-phenoxybenzyl ether, dimethyl-(4-ethoxyphenyl)-silylmethyl-3-phenoxybenzyl ether, dimethylvinphos, dioxathion, disulfoton,
eflusilanate, emamectin, empenthrin, endosulfan, EPN, esfenvalerate, ethiofencarb, ethion, ethofenprox, etrimphos, etoxazole, etobenzanid,
fenamiphos, fenazaquin, fenbutatin oxide, fenfluthrin, fenitrothion, fenobucarb, fenothiocarb, fenoxycarb, fenpropathrin, fenpyrad, fenpyroximat, fensulfothion, fenthion, fenvalerate, fipronil, flonicamid, fluacrypyrim, fluazuron, flucycloxuron, flucythrinate, flufenerim, flufenoxuron, flupyrazofos, flufenzine, flumethrin flufenprox, fluvalinate, fonophos, formethanate, formothion, fosmethilan fosthiazate, fubfenprox, furathiocarb,
halofenocid, HCH, (CAS RN: 58-89-9), heptenophos, hexaflumuron, hexythiazox, hydramethylnon, hydroprene,
imidacloprid, imiprothrin, indoxycarb, iodfenfos, iprinomectin, iprobenfos, isazophos, isoamidophos, isofenphos, isoprocarb, isoprothiolane, isoxathion, ivermectin, lambda-cyhalothrin, lufenuron,
kadedrin
lambda-cyhalothrin, lufenuron,
malathion, mecarbam, mervinphos, mesulfenphos, metaldehyde, methacrifos, methamidophos, methidathion, methiocarb, methomyl, metalcarb, milbemectin, monocrotophos, moxiectin,
naled, NI 125, nicotine, nitenpyram, noviflumuron,
omethoate, oxamyl, oxydemethon M, oxydeprofos,
parathion A, parathion M, penfluron, permethrin, 2-(4-phenoxyphenoxy)-ethyl ethylcarbamate, phenthoate, phorate, phosalon, phosmet, phosphamidon, phoxim, pirimicarb, pirimiphos M, pirimiphos A, prallethrin, profenophos, promecarb, propaphos, propoxur, prothiophos, prothoate, pymetrozin, pyrachlophos, pyridaphenthion, pyresmethrin, pyrethrum, pyridaben, pyridalyl, pyrimidifen, pyriproxifen, pyrithiobac-sodium
quinalphos,
resmethrin, rotenone,
salithion, sebufos, silafluofen, spinosad, spirodiclofen, spiromesifen, sulfotep, sulprofos,
tau-fluvalinate, taroils, tebufenozide, tebufenpyrad, tebupirimphos, teflubenzuron, tefluthrin, temephos, terbam, terbufos, tetrachlorvinphos, tetramethrin, Tetramethacarb, thiacloprid, thiafenox, thiamethoxam, thiapronil, thiodicarb, thiofanox, thiazophos, thiocyclam, thiomethon, thionazin, thuringiensin, tralomethrin, transfluthrin, triarathen, triazophos, triazamate, triazuron, trichlorfon, triflumuron, trimethacarb,
vamidothion, xylylcarb, zetamethrin;
   molluscicides:
fentin acetate, metaldehyde, methiocarb, niclosamide;
   herbicides and algicides:
acetochlor, acifluorfen, aclonifen, acrolein, alachlor, alloxydim, ametryn, amidosulfuron, amitrole, ammonium sulfamate, anilofos, asulam, atrazine, azafenidin, aziptrotryne, azimsulfuron,
benazolin, benfluralin, benfuresate, bensulfuron, bensulfide, bentazone, benzofencap, benzthiazuron, bifenox, bispyribac, bispyribac-sodium, borax, bromacil, bromobutide, bromofenoxim, bromoxynil, butachlor, butamifos, butralin, butylate, bialaphos, benzoyl-prop, bromobutide, butroxydim,
carbetamide, carfentrazone-ethyl, carfenstrole, chlomethoxyfen, chloramben, chlorbromuron, chlorflurenol, chloridazon, chlorimuron, chlomitrofen, chloroacetic acid, chloransulam-methyl, cinidon-ethyl, chlorotoluron, chloroxuron, chlorpropham, chlorsulfuron, chlorthal, chlorthiamid, cinmethylin, cinofulsuron, clefoxydim, clethodim, clomazone, chlomeprop, clopyralid, cyanamide, cyanazine, cycloate, cycloxydim, chloroxynil, clodinafop-propargyl, cumyluron, clometoxyfen, cyhalofop, cyhalofop-butyl, clopyrasuluron, cyclosulfamuron,
diclosulam, dichlorprop, dichlorprop-P, diclofop, diethatyl, difenoxuron, difenzoquat, diflufenican, diflufenzopyr, dimefuron, dimepiperate, dimethachlor, dimethipin, dinitramine, dinoseb, dinoseb acetate, dinoterb, diphenamid, dipropetryn, diquat, dithiopyr, diduron, DNOC, DSMA, 2,4-D, daimuron, dalapon, dazomet, 2,4-DB, desmedipham, desmetryn, dicamba, dichlobenil, dimethamid, dithiopyr, dimethametryn,
eglinazine, endothal, EPTC, esprocarb, ethalfluralin, ethidimuron, ethofumesate, ethobenzanid, ethoxyfen, ethametsulfuron, ethoxysulfuron,
fenoxaprop, fenoxaprop-P, fenuron, flamprop, flamprop-M, flazasulfuron, fluazifop, fluazifop-P, fuenachlor, fluchloralin, flufenacet flumeturon, fluorocglycofen, fluoronitrofen, flupropanate, flurenol, fluridone, flurochloridone, fluroxypyr, fomesafen, fosamine, fosametine, flamprop-isopropyl, flamprop-isopropyl-L, flufenpyr, flumiclorac-pentyl, flumipropyn, flumioxzim, flurtamone, flumioxzim, flupyrsulfuron-methyl, fluthiacet-methyl,
glyphosate, glufosinate-ammonium
haloxyfop, hexazinone,
imazamethabenz, isoproturon, isoxaben, isoxapyrifop, imazapyr, imazaquin, imazethapyr, ioxynil, isopropalin, imazosulfuron, imazomox, isoxaflutole, imazapic,
ketospiradox,
lactofen, lenacil, linuron,
MCPA, MCPA-hydrazide, MCPA-thioethyl, MCPB, mecoprop, mecoprop-P, mefenacet, mefluidide, mesosulfuron, metam, metamifop, metamitron, metazachlor, methabenzthiazuron, methazole, methoroptryne, methyldymron, methyl isothiocyanate, metobromuron, metoxuron, metribuzin, metsulfuron, molinate, monalide, monolinuron, MSMA, metolachlor, metosulam, metobenzuron,
naproanilide, napropamide, naptalam, neburon, nicosulfuron, norflurazon, sodium chlorate,
oxadiazon, oxyfluorfen, oxysulfuron, orbencarb, oryzalin, oxadiargyl,
propyzamide, prosulfocarb, pyrazolate, pyrazolsulfuron, pyrazoxyfen, pyribenzoxim, pyributicarb, pyridate, paraquat, pebulate, pendimethalin, pentachlorophenol, pentoxazone, pentanochlor, petroleum oils, phenmedipham, picloram, piperophos, pretilachlor, primisulfuron, prodiamine, profoxydim, prometryn, propachlor, propanil, propaquizafob, propazine, propham, propisochlor, pyriminobac-methyl, pelargonic acid, pyrithiobac, pyraflufen-ethyl,
quinmerac, quinocloamine, quizalofop, quizalofop-P, quinchlorac,
rimsulfuron
sethoxydim, sifuron, simazine, simetryn, sulfosulfuron, sulfometuron, sulfentrazone, sulcotrione, sulfosate,
tar oils, TCA, TCA-sodium, tebutam, tebuthiuron, terbacil, terbumeton, terbutylazine, terbutryn, thiazafluoron, thifensulfuron, thiobencarb, thiocarbazil, tralkoxydim, triallate, triasulfinuron, tribenuron, triclopyr, tridiphane, trietazine, trifluoralin, tycor, thdiazimin, thiazopyr, triflusulfuron,
vernolate.

The weight ratios of the active compounds in these active compound combinations can be varied within relatively wide ranges.

Preferably, the active compound combinations comprise the active compound of the general formula (I) in an amount of from 0.1 to 99.9%, in particular from 1 to 75%, especially preferably from 5 to 50%, the remainder to 100% being one or more of the co-components mentioned above.

The microbicidal compositions or concentrates used for protecting the industrial materials comprise the active compound of the general formula (I) or the active compound combination of the active compound of the formula (I) with one of the co-components mentioned above in a concentration of 0.01 and 95% by weight, in particular from 0.1 to 60% by weight.

The use concentrations of the active compounds or active compound combinations to be used depend on the nature and the occurrence of the microorganisms to be controlled and on the composition of the material to be protected. The optimum rate of application can be determined by test series. In general, the use concentrations are in the range from 0.001 to 5% by weight, preferably from 0.05 to 1.0% by weight, based on the material to be protected.

With the active compounds or compositions according to the invention, it is possible to replace, in an advantageous manner, the microbicidal compositions available to date by more effective compositions. They have good stability and, in an advantageous manner, a broad activity spectrum.

### EXAMPLES

### Example 1

### 2-(Hydroxyamino)-1-cyclopentene-1-carbonitrile

of the formula

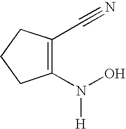

2.00 g (0.019 mol) of 2-amino-1-cyclopentene-1-carbonitrile and 1.29 g (0.0187 mol) of hydroxylamine hydrochloride were suspended in 10 ml of ethanol, and the mixture was heated at the boil for 4 hours. After cooling, the solid was filtered off and the filtrate was concentrated. The residue that remained was recrystallized from toluene/hexane=5/1. This gave 1.38 g (57% of theory) of the title compound as a light-brown solid of m.p.=84° C.

### Example 2

### 2-(tert-Butoxyamino)-1-cyclopentene-1-carbonitrile

of the formula

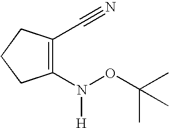

0.94 g (0.008 mol) of O-tert-butylhydroxylamine hydrochloride was added to a solution of 0.81 g (0.008 mol) of 2-amino-1-cyclopentene-1-carbonitrile in 10 ml of ethanol, and the mixture was heated at the boil for two hours. After cooling, the solid formed was filtered off and the filtrate was concentrated. Drying of the residue under reduced pressure gave 1.05 g (74% of theory) of the title compound as a colorless oil of n_{D}²⁴=1.4590.

### Example 3

### 2-[(Benzyloxy)amino]-1-cyclopentene-1-carbonitrile

of the formula

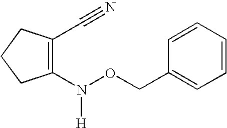

0.94 g (0.01 mol) of O-benzylhydroxylamine hydrochloride was added to a solution of 1.08 g (0.01 mol) of 2-amino-1-cyclopentene-1-carbonitrile in 10 ml of ethanol, and the mixture was heated at the boil for two hours. After cooling, the solid formed was filtered off and the filtrate was concentrated. Drying of the residue under reduced pressure gave 1.72 g (76% of theory) of the title compound as a colorless oil of n_{D}²⁴=1.4721.

### Example 4

### N-(2-Cyano-1-cyclopenten-1-yl)-N-(isobutyryloxy)-2-methylpropanamide

of the formula

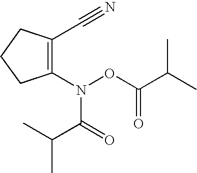

At 0° C., 4.37 g (0.04 mol) of isobutyryl chloride were added dropwise over a period of one hour to a solution of 4.97 g (0.04 mol) of 5,6-dihydro-4H-cyclopenta[c]isoxazole-3-amine and 9.50 g (0.09 mol) of triethylamine in 50 ml of THF, and the mixture was then allowed to warm to room temperature. The mixture was then heated at the boil for 4 hours. After cooling, the reaction mixture was introduced into 300 ml of water and extracted four times with in each case 100 ml of dichloromethane. The combined organic phases were dried over sodium sulfate and concentrated under reduced pressure, and the residue that remained was chromatographed on silica gel (toluene/ethyl acetate=10/1). This gave 3.20 g (29% of theory) of the title compound as a pale yellow oil of n_{D}²⁴=1.4965.

The compounds listed in Table 1 can be obtained analogously to Examples 1, 2, 3 and 4 and/or in accordance with the general statements in the descriptions of the experiments.

TABLE 1

| Example | Structural formula | Physical data |
|---|---|---|
| 5 | 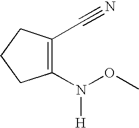 | n_{D}²⁴ = 1.4708 |
| 6 | 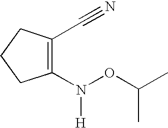 | n_{D}²⁴ = 1.4645 |
| 7 | 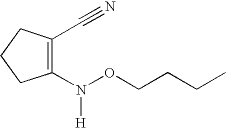 | n_{D}²² = 1.4712 |
| 8 | 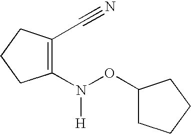 | n_{D}²⁵ = 1.4867 |
| 9 | 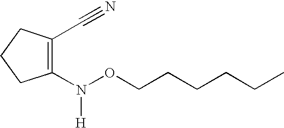 | n_{D}²³ = 1.4635 |
| 10 | 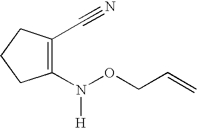 | n_{D}²⁴ = 1.4196 |
| 11 | 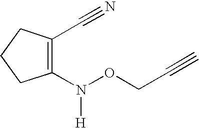 | n_{D}²³ = 1.4613 |
| 12 | 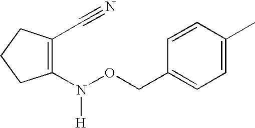 | n_{D}²⁴ = 1.4354 |
| 13 | 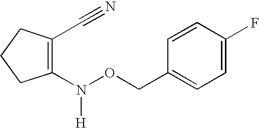 | n_{D}²⁴ = 1.4786 |
| 14 | 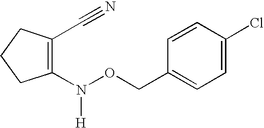 | n_{D}²⁴ = 1.4325 |
| 15 | 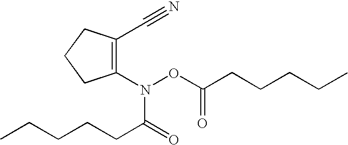 | n_{D}²³ = 1.4935 |

### Example 16

### 5,6-Dihydro-4H-cyclopenta[c]isoxazole-3-amine

of the formula

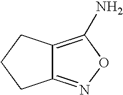

34.75 g (0.5 mol) of hydroxylamine hydrochloride were added to a solution of 54.07 g (0.5 mol) of 2-amino-1-cyclopentene-1-carbonitrile in 250 ml of ethanol, and the mixture was heated at the boil for one hour. The precipitate formed was filtered off, and the filtrate was concentrated and then taken up in a mixture of 60 ml of isopropanol and 240 ml of water. The mixture was heated at 40° C., and 0.05 mol of 50 percent strength aqueous sodium hydroxide solution was added dropwise at this temperature. The mixture was then stirred at 60° C. for another half an hour and then cooled to 10° C., and the solid formed was filtered off. Washing with water and drying gave 51.4 g (82% of theory) of the title compound as a white solid of m.p.=135° C.

### Use Example A

To demonstrate the activity against bacteria, the minimum inhibitory concentrations (MIC) of compounds according to the invention were determined:

A defined Landy Agar was admixed with the active compounds according to the invention in concentrations of from 0.1 mg/ml to 5000 mg/ml. After the agar had solidified, it was contaminated with pure cultures of the test organisms listed in Table 2. The MIC was determined after 3 days of storage at 28° C. and 60-70% relative atmospheric humidity. The MIC is the lowest concentration of active compound at which there is no colonization by the microbial species used, it is stated in the table below.

TABLE 2
Minimum inhibitory concentrations (ppm) of compounds
of the formula (I) according to the invention

| Example No. | Pseudomonas aeroginosa | Bacillus subtilis |
|---|---|---|
| 1 | <400 | <400 |
| 4 | <400 | <400 |

### Use Example B

To demonstrate the activity against fungi, the minimum inhibitory concentrations (MIC) of compositions according to the invention were determined:

An agar which had been prepared using malt extract was admixed with active compounds according to the invention in concentrations of from 0.1 mg/l to 5000 mg/l. After the agar had solidified, it was contaminated with pure cultures of the test organisms listed in Table 3. The MIC was determined after 2 weeks of storage at 28° C. and 60 to 70% relative atmospheric humidity. The MIC is the lowest concentration of active compound at which there is no colonization by the microbial species used, it is stated in Table 3 below.

TABLE 3
Minimum inhibitory concentrations (ppm) of compounds
of the formula (I) according to the invention

| Example No. | Penicillium brevicaule | Chaetomium globosum |
|---|---|---|
| 3 | <400 | <400 |
| 4 | <400 | <400 |
| 12 | <400 | <400 |
| 12 | <400 | <400 |
| 14 | <400 | <400 |

1. A compound of the formula (I) 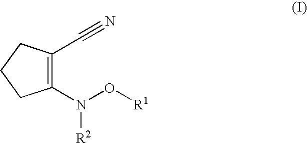
in which
R¹ and R² independently of one another represent hydrogen, halogen, cyano, nitro or represent in each case optionally substituted alkyl, alkenyl, alkynyl, aryl, heterocyclyl or —COR³,
where
R³ represents in each case optionally substituted alkyl, alkenyl, alkynyl, aryl or heterocyclyl,
or a salt or an acid addition compound thereof.

2. The compound as claimed in claim 1, characterized in that
R¹ and R² independently of one another represent hydrogen, halogen, cyano, nitro or in each case optionally substituted C₁-C₈-alkyl, C₂-C₈-alkenyl, C₂-C₈-alkynyl, phenyl or heterocyclyl, or represent a radical —COR³,
where
R³ represents hydrogen, halogen, cyano, nitro or represents in each case optionally substituted C₁-C₈-alkyl, C₂-C₈-alkenyl, C₂-C₈-alkynyl, phenyl or heterocyclyl.

3. A process for preparing compounds of the formula (I) as claimed in claim 1,
where R¹ and R² are as defined in claim 1,
except for compounds of the formula (I) in which R¹ and R² are identical and represent —COR³,
characterized in that 2-amino-1-cyclopentene-1-carbonitrile of the formula 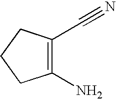
is reacted with hydroxylamines of the general formula (II)
or salts thereof, 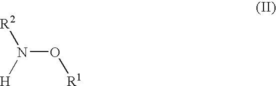
in which R¹ and R² are as defined in claim 1,
but R¹ and R² do not simultaneously represent —COR³,
if appropriate in the presence of diluents and if appropriate in the presence of a catalytic or stoichiometric amount of base.

4. A process for preparing compounds of the general formula (I) as claimed in claim 1,
where R¹ and R² are identical and represent —COR³,
characterized in that 5,6-dihydro-4H-cyclopenta[c]isoxazol-3-amine of the formula 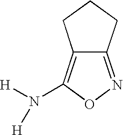
is reacted with carbonyl chlorides of the general formula (III) 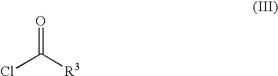
where R³ is as defined in claim 1,
if appropriate in the presence of diluents and if appropriate in the presence of a catalytic or stoichiometric amount of base.

5. A microbicidal composition, comprising at least one compound as claimed in at least one of claims 1 and 2 and at least one solvent or diluent and also, if appropriate, processing auxiliaries and, if appropriate, further antimicrobially active compounds.

6. The composition as claimed in claim 5, characterized in that it comprises at least one further antimicrobially active compound from the group of the fungicides, bactericides, herbicides and/or insecticides.

7. The use of compounds as claimed in at least one of claims 1 and 2 as microbicide for protecting industrial materials.

8. The use as claimed in claim 7, characterized in that the industrial materials are adhesives, sizes, paper, cardboard, leather, wood, timber products, paints, cooling lubricants and heat transfer fluids.

9. A method for protecting industrial materials against attack and/or destruction by microorganisms, characterized in that at least one compound as claimed in at least one of claims 1 and 2 is allowed to act on the microorganism or its habitat.

10. An industrial material which comprises at least one compound as claimed in at least one of claims 1 and 2.
